(19) United States
(12) Patent Application Publication
(10) Pub. No.: US 2025/0255910 A1
(43) Pub. Date: Aug. 14, 2025

(54) USE OF STAPHYLOCOCCUS LENTUS IN PREPARATION OF COMPOSITION

(71) Applicant: BEIJING FRIENDSHIP HOSPITAL, CAPITAL MEDICAL UNIVERSITY, Beijing (CN)

(72) Inventors: Dan YAN, Beijing (CN); Jianglan LONG, Beijing (CN); Aiting WANG, Beijing (CN)

(73) Assignee: BEIJING FRIENDSHIP HOSPITAL, CAPITAL MEDICAL UNIVERSITY, Beijing (CN)

(21) Appl. No.: 19/112,994

(22) PCT Filed: Jun. 9, 2023
(86) PCT No.: PCT/CN2023/099382

§ 371 Date: Mar. 19, 2025

(30) Foreign Application Priority Data
Sep. 21, 2022 (CN) 202211146734.1

(51) Int. Cl. A61K 35/741; C12N 1/20; C12R 1/44

(52) U.S. Cl. CPC A61K 35/741; C12N 1/205; C12R 2001/44

(57) ABSTRACT

Provided is a use of Staphylococcus lentus with an accession number of GDMCC 1.247 in preparation of a composition for preventing and treating insulin resistance or type 2 diabetes. This strain can significantly reduce the weight gain induced by a high-fat diet (HFD), improve the glucose tolerance, promote the insulin secretion, improve the insulin resistance, and significantly improve the immunity and inflammation.

### CROSS REFERENCE TO THE RELATED APPLICATIONS

This application is the national phase entry of International Application No. PCT/CN2023/099382, filed on Jun. 9, 2023, which is based upon and claims priority to Chinese Patent Application No. 202211146734.1, filed on Sep. 21, 2022, the entire contents of which are incorporated herein by reference.

### TECHNICAL FIELD

The present disclosure belongs to the fields of food and biomedicine, and specifically relates to a use of Staphylococcus lentus in preparation of a composition for preventing and treating an insulin resistance symptom and type 2 diabetes.

### BACKGROUND

Insulin resistance is an outstanding characteristic for type 2 diabetes and is also one of the basic links in the pathogenesis for type 2 diabetes. The incidence of type 2 diabetes is increasing year by year, and type 2 diabetes is a disease difficult to cure. Insulin resistance can be cured through early intervention and lifestyles such as strict diet control and exercise enhancement. The effective prevention and treatment of insulin resistance and the reduction of a transformation rate from insulin resistance to type 2 diabetes can reduce the prevalence of type 2 diabetes. Insulin resistance, or type 2 diabetes, is often accompanied by weight gain, impaired glucose tolerance, immune weakness, and low-grade inflammation. Therefore, the improvement of weight gain, impaired glucose tolerance, immunity, and inflammation is a way to prevent and treat insulin resistance or type 2 diabetes.

Gut microbiota are closely related to the occurrence and development of insulin resistance or type 2 diabetes. The abundance of Staphylococcus lentus among many gut microbiota decreases with the occurrence and development of insulin resistance or type 2 diabetes (Gut bacteria responding to dietary change encode sialidases that exhibit preference for red meat-associated carbohydrates. Nat Microbiol. 2019, 4: 2082-2089; Effects of oral sialic acid on gut development, liver function and gut microbiota in mice. Lett Appl Microbiol. 2021, 73: 20-25). However, it is unclear whether Staphylococcus lentus has the biological function of resisting insulin.

In view of this, the present disclosure is specifically proposed.

### SUMMARY

An objective of the present disclosure is to provide a use of Staphylococcus lentus in preparation of a composition for preventing and treating an insulin resistance symptom.

To achieve the above objective, the following technical solutions are adopted:

The present disclosure provides a use of Staphylococcus lentus in preparation of a composition, where the composition is a food, a health care product, or a drug, and the composition is used to prevent or treat insulin resistance or type 2 diabetes.

Optionally, the product is a food, a health care product, or a drug.

Optionally, according to the use described above, the Staphylococcus lentus is a strain deposited in Guangdong Microbial Culture Collection Center (GDMCC), with an accession number of GDMCC 1.247 and a deposit date of Jan. 1, 2005; and the address of GDMCC is 5th Floor, Experimental Building, No. 100, Xianlie Middle Road, Yuexiu District, Guangzhou, Guangdong, China.

Optionally, according to the use described above, the Staphylococcus lentus is in a viable cell form.

Optionally, according to the use described above, the drug includes the Staphylococcus lentus as a major active ingredient.

Optionally, the type 2 diabetes is induced by the insulin resistance.

Optionally, the insulin resistance is caused by a high-fat diet (HFD).

Optionally, the use described above further includes improving one or more of symptoms such as weight gain, impaired glucose tolerance, immune weakness, and inflammation.

Further, the symptoms are caused by HFD.

Further, the improving immune weakness refers to increasing immunoglobulin A, immunoglobulin G, and interferon-γ in an individual.

Further, the improving inflammation refers to reducing a pro-inflammatory factor concentration and increasing an anti-inflammatory factor concentration in an individual on HFD. In a specific embodiment, the improving inflammation refers to reducing the secretion of lipopolysaccharides and promoting the synthesis and secretion of interleukin 10 in an individual on HFD.

Optionally, according to the use described above, the health care product or the drug is a liquid preparation or a solid preparation.

Optionally, the drug is an oral drug. Optionally, the drug is administered based on a gastrointestinal tract. Optionally, the gastrointestinal administration may refer to the administration based on parts such as an esophagus, a stomach, a small intestine, a colon, and a rectum. Optionally, the drug is a powder, a tablet, a granule, a capsule, a solution, an emulsion, a suspension, etc. For example, in a specific embodiment, the drug is a solution, which is administered intragastrically. In some embodiments, the drug is a capsule, which is administered orally. In some embodiments, the drug is a solution or a suspension, which is administered through a small intestine, a colon, or a rectum.

The present disclosure has the following beneficial effects:

The Staphylococcus lentus of the present disclosure can significantly reduce the weight gain induced by HFD, improve the glucose tolerance, promote the insulin secretion, improve the insulin resistance, and significantly improve the immunity and inflammation. The Staphylococcus lentus provided by the present disclosure can effectively prevent and treat the insulin resistance with high safety and no obvious adverse reactions. Thus, the Staphylococcus lentus has a promising prospect.

### BRIEF DESCRIPTION OF THE DRAWINGS

FIG. 1 shows the body weights of mice intervened for 6 weeks;

FIG. 2 shows the oral glucose tolerance test (OGTT) results for mice after intervention;

FIG. 3 shows the 2-hour postprandial blood glucose values of mice after intervention;

FIG. 4 shows the insulin tolerance test (ITT) results for mice after intervention;

FIG. 5 shows the quantification results of cumulative blood glucose changes in mice after intervention based on the area under the ITT curve (AUC of ITT);

FIG. 6 shows the insulin concentrations in mice after intervention;

FIG. 7 shows the homeostatic model assessment for insulin resistance (HOMA-IR) results of mice after intervention;

FIG. 8 shows the alanine aminotransferase concentrations in mice after intervention;

FIG. 9 shows the aspartate aminotransferase concentrations in mice after intervention;

FIG. 10 shows the total cholesterol concentrations in mice after intervention;

FIG. 11 shows the high-density lipoprotein cholesterol concentrations in mice after intervention;

FIG. 12 shows the immunoglobulin A concentrations in mice after intervention;

FIG. 13 shows the immunoglobulin G concentrations in mice after intervention;

FIG. 14 shows the serum interferon-γ concentrations in mice after intervention;

FIG. 15 shows the serum interleukin 1β concentrations in mice after intervention;

FIG. 16 shows the serum lipopolysaccharide concentrations in mice after intervention; and

FIG. 17 shows the serum interleukin 10 concentrations in mice after intervention.

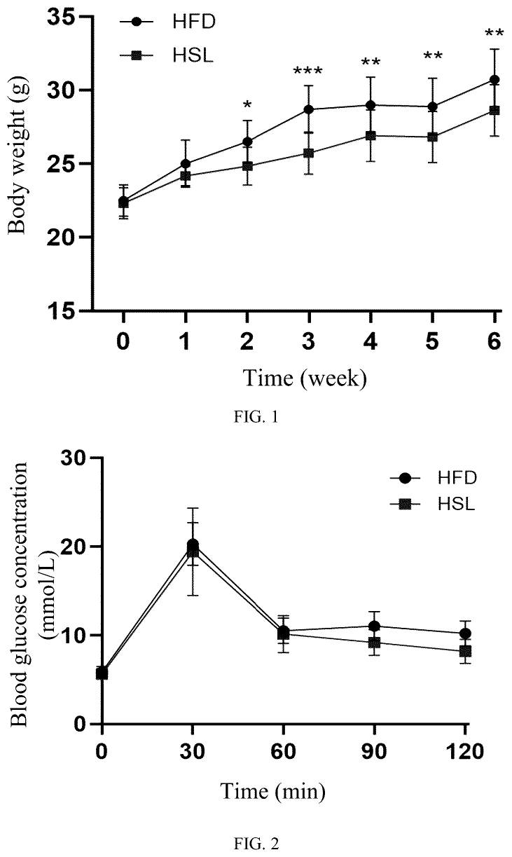

In FIG. 1 to FIG. 15, HFD and HSL represent high-fat diet and high-fat diet+Staphylococcus lentus, respectively; there are 8 mice in each group; and *P<0.05, **P<0.01, ***P<0.001, and ****P<0.0001 indicate statistically-significant differences compared with the HFD group.

### DETAILED DESCRIPTION OF THE EMBODIMENTS

In order to further elaborate the technical means and results adopted by the present disclosure to achieve the intended objective of the present disclosure, the specific embodiments, technical solutions, and features of the present disclosure are described in detail below through preferred examples. Specific features, structures, or characteristics in various examples described below may be combined in any appropriate form.

The main materials adopted in the following examples of the present disclosure and sources thereof are as follows:

Staphylococcus lentus is a strain deposited in Guangdong Microbial Culture Collection Center (GDMCC), with an accession number of GDMCC 1.247 and a deposit date of Jan. 1, 2005, which is purchased from NingBo Testobio Co., Ltd. A nutrient broth medium (Cat. No.: N8300) and glucose (Cat. No.: G8150) are purchased from Beijing Solarbio Science & Technology Co., Ltd. A basal feed (Batch No.: 21103313) is purchased from Beijing Keao Xieli Feed Co., Ltd. A high-fat feed (Cat. No.: D12492, 60% of calories come from fats) is purchased from Research diets, USA. An insulin injection is purchased from Fosun Wanbang Pharma Group. A total cholesterol test kit (Cat. No.: A111-1-1), a high-density lipoprotein cholesterol test kit (Cat. No.: A112-1-1), an alanine aminotransferase test kit (Cat. No.: C009-2-1), an aspartate aminotransferase test kit (Cat. No.: C010-2-1), and a glucose test kit (Cat. No.: A154-1-1) are purchased from Nanjing Jiancheng Bioengineering Institute. An insulin ELISA kit (Cat. No.: F5618B), a lipopolysaccharide ELISA kit (Cat. No.: F10621B), an interleukin 1β ELISA kit (Cat. No.: F5079B), an interleukin 10 ELISA kit (Cat. No.: F5215B), an immunoglobulin A ELISA kit (Cat. No.: F5094B), an immunoglobulin G ELISA kit (Cat. No.: F5096B), and an interferon-γ ELISA kit (Cat. No.: F5221B) all are purchased from Jiangsu Meimian Industrial Co., Ltd. 1.5 mL centrifuge tubes (Axygen, USA). Glucometer (Roche Diagnostics, Shanghai). Multi-purpose vortex mixer (Vortex Genie 2, Scientific Industries, USA). Refrigerated high-speed centrifuge (Sigma, Germany). Biochemical incubator (Shanghai Yiheng Instruments Co., Ltd.). Multimode microplate reader (BioTek, USA).

### Example 1: Preparation of Staphylococcus lentus Bacterial Solution for Intragastric Administration

18.0 g of a nutrient broth was weighed, added to 1,000 mL of ultrapure water, heated and boiled for dissolution, dispensed, sealed with a filter membrane, and autoclaved for 30 min for later use.

A glycerol-preserved culture stored in a freezer at −80° C. was taken out, immediately placed in a 37° C. to 45° C. water bath for quick thawing, and shaken appropriately until the glycerol-preserved culture in a glycerol tube was completely thawed. The glycerol tube was opened aseptically, and the culture was transferred by a sterile pipette to a freshly-prepared medium, cultured, and passaged twice for later use.

15 mL of the nutrient broth medium was taken, and 50 μL of a Staphylococcus lentus bacterial solution was inoculated in the nutrient broth medium and cultured in the biochemical incubator at 37° C. and 250 rpm for 5 h. OD₆₂₅ of a resulting bacterial solution was detected by a microplate reader to be 0.30 to 0.39, that is, the bacterial solution had a concentration of 4.5×10⁸ CFU/mL (CFU represents Colony-forming Unit).

### Example 2: Staphylococcus lentus Could Reduce the Weight Gain Induced by HFD

16 male C57BL/6J mice at 6 weeks (experimental animal license No.: SCXK (Beijing) 2019-0009) were purchased from Beijing Vital River Laboratory Animal Technology Co., Ltd. Raising conditions: Specific Pathogen Free (SPF) level cleanliness, room temperature: 20° C. to 22° C., humidity: 60±5%, 12 h light/dark cycle, and free eating and drinking. All animal protocols in this experiment were approved by the Beijing MaideiKangna Animal Ethics Committee, and animal experimental operations were conducted under the guidance of the Laboratory Animal Protection Association.

After being adaptively raised for 7 d with the basal feed, the mice were weighed. According to body weights (g), the mice were randomly grouped into an HFD group and an HFD+Staphylococcus lentus (HSL) group, with 8 mice in each group. Then the mice all were raised with a high-fat feed for 6 weeks. During the raising period, the HFD group was intragastrically administered with the nutrient broth medium at 0.20 mL/mouse daily, and the HSL group was intragastrically administered with the Staphylococcus lentus bacterial solution at 0.20 mL/mouse daily (that is, 9×10⁷ CFU/d of Staphylococcus lentus). During the raising period, a body weight of each mouse was monitored weekly.

Results were shown in FIG. 1. Mice in the HSL group had a significantly-lower body weight than mice in the HFD group at week 3 after intervention, indicating that the treatment with Staphylococcus lentus could significantly reduce the HFD-induced weight gain.

### Example 3: Staphylococcus lentus Could Improve the Impaired Glucose Tolerance and Insulin Sensitivity That Were Induced by HFD

6 weeks after intervention, OGTT was conducted for mice in the HFD group and HSL group to investigate the influence of Staphylococcus lentus on the glucose tolerance of HFD-induced mice. At week 6 after administration, mice in each group were fasted for 12 h without water deprivation. Then the mice were weighed, and blood was collected from the tail and tested for a fasting blood glucose value (before glucose load). Then a 20% glucose solution was intragastrically administered at a dose of 2.0 g/kg. At 30 min, 60 min, 90 min, and 120 min (after glucose load), blood was collected from tail tips of experimental mice and tested with a glucose strip and a glucometer for a blood glucose value.

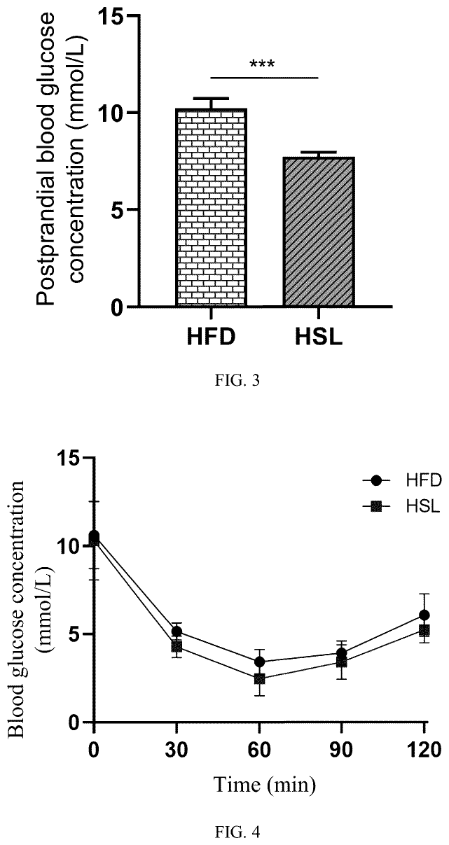

Results were shown in FIG. 2 to FIG. 3. Although there was no difference in the AUC of OGTT (FIG. 2) between the groups, the HSL group had a significantly-lower 2-hour postprandial blood glucose value than the HFD group (FIG. 3), indicating that Staphylococcus lentus could improve the HFD-induced impaired glucose tolerance.

Moreover, ITT was conducted to investigate the influence of Staphylococcus lentus on the insulin sensitivity in HFD-induced mice. At week 6 after intragastric administration, mice in each group were fasted for 6 h without water deprivation. Then the mice were weighed, and blood was collected from the tail and tested for a fasting blood glucose value (before insulin injection). Then insulin was intraperitoneally injected (0.5 U/kg). At 30 min, 60 min, 90 min, and 120 min (after insulin injection), blood was collected from tail tips of mice and tested with a glucose strip and a glucometer for a blood glucose value.

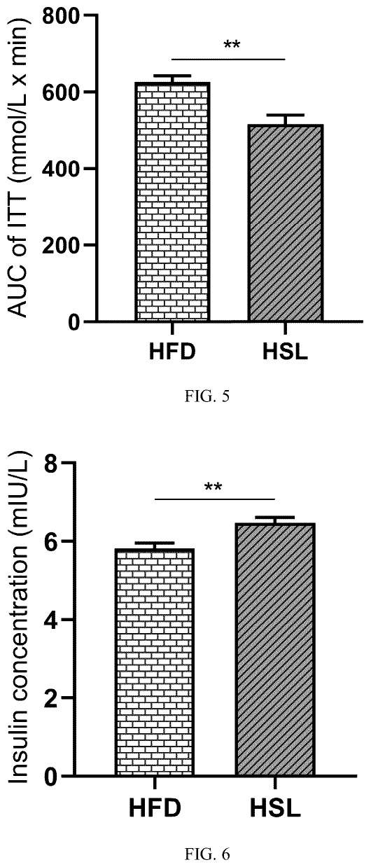

Results were shown in FIG. 4 to FIG. 5. Mice in the HSL group had a significantly-smaller AUC of ITT than mice in the HFD group (P<0.01), indicating that Staphylococcus lentus could improve the insulin sensitivity of HFD-induced mice.

### Example 4: Staphylococcus lentus Could Promote the Insulin Secretion and Improve the Insulin Resistance

After 6 weeks of intervention, mice in each group were fasted for 12 h without water deprivation. Then blood was collected from the orbital venous plexus, allowed to stand at room temperature for 30 min to 60 min, and centrifuged at 4° C. and 16,200 g for 10 min. A resulting supernatant was collected, dispensed in polypropylene EP tubes, and stored at −80° C. for the subsequent detection of observation indexes.

A serum insulin concentration in each mouse was detected according to steps of the ELISA kit. Results were shown in FIG. 6. Mice in the HSL group had a significantly-higher insulin concentration than mice in the HFD group (P<0.01), indicating that Staphylococcus lentus could promote the insulin secretion.

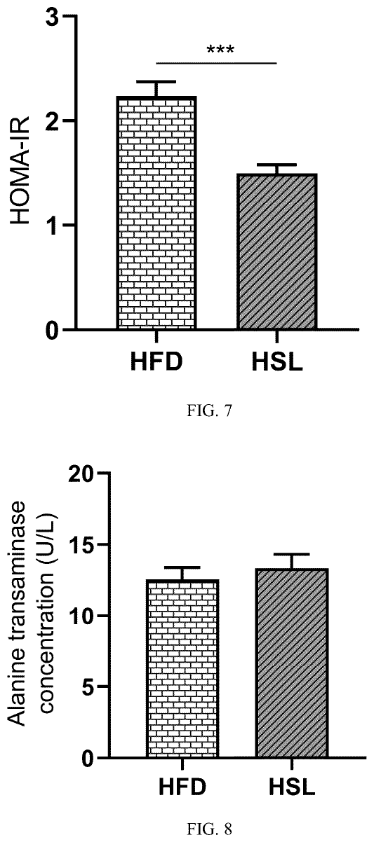

Serum glucose concentrations of mice in each group were detected with a glucose kit, and HOMA-IR was calculated accordingly: HOMA-IR=blood glucose concentration (mmol/L)×insulin concentration (mIU/L)/22.5. HOMA-IR is often used to assess the degree of insulin resistance for an individual. Results were shown in FIG. 7. Mice in the HFD group had significantly-higher HOMA-IR than mice in the HSL group (P<0.001), indicating that Staphylococcus lentus could effectively improve the HFD-induced insulin resistance.

### Example 5: Influence of Staphylococcus lentus on the Liver Function and Lipid Metabolism in Mice

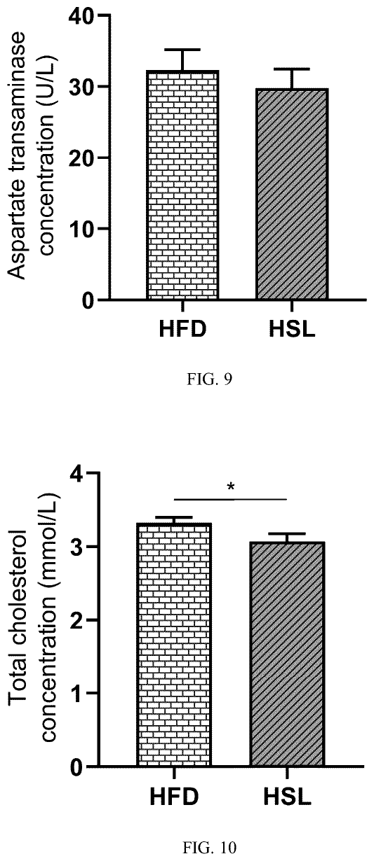

Serum alanine aminotransferase and aspartate aminotransferase concentrations in mice were detected according to steps of a kit to investigate the influence of Staphylococcus lentus on the liver function in mice. Results were shown in FIG. 8 to FIG. 9. Alanine aminotransferase concentrations (FIG. 8) and aspartate aminotransferase concentrations (FIG. 9) of the HFD group and the HSL group were not different from each other, and both were in the normal ranges, indicating that Staphylococcus lentus had no influence on the liver function in mice.

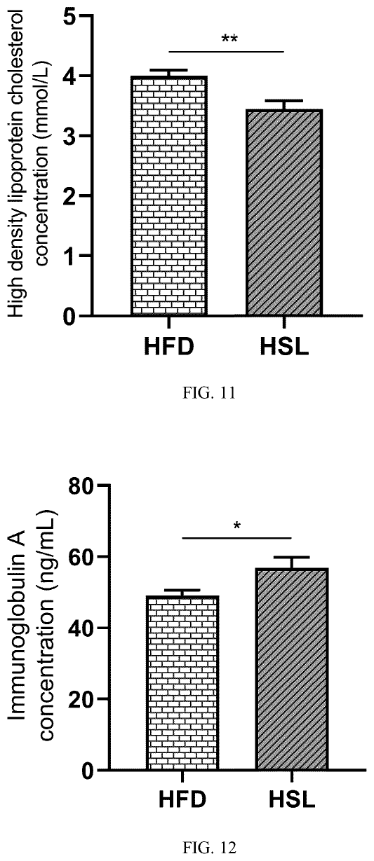

Serum total cholesterol and high-density lipoprotein cholesterol concentrations in mice were detected according to steps of kits to investigate the influence of Staphylococcus lentus on the lipid metabolism in mice. Results were shown in FIG. 10 to FIG. 11. Total cholesterol (FIG. 10) and high-density lipoprotein cholesterol (FIG. 11) concentrations in mice of the HSL group were significantly lower than total cholesterol and high-density lipoprotein cholesterol concentrations in mice of the HFD group, indicating that Staphylococcus lentus could regulate the lipid metabolism disorders induced by HFD.

### Example 6: Staphylococcus lentus Could Improve the Immunity and Inflammation in Mice

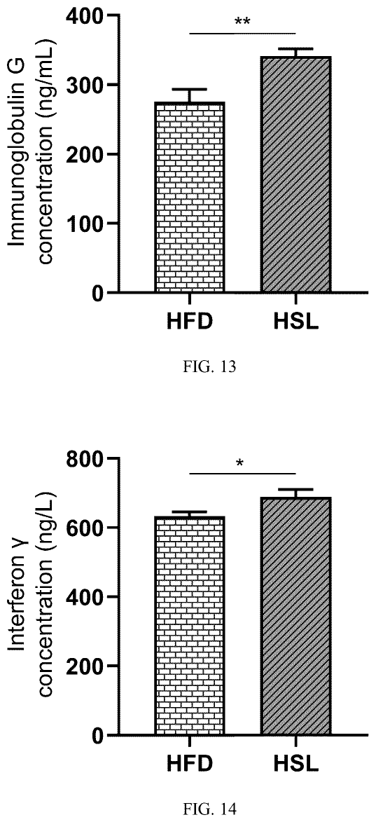

Immunoglobulin A is not only an important immunomodulator, but also an important regulator for blood glucose homeostasis. Interferon-γ is a lymphokine with an extensive immunomodulatory effect. Immunoglobulin A, immunoglobulin G, and interferon-γ concentrations in mice were detected according to steps of ELISA kits to investigate the influence of Staphylococcus lentus on the immunity (including humoral immunity and cellular immunity) of mice. Results were shown in FIG. 12 to FIG. 14. Immunoglobulin A (FIG. 12), immunoglobulin G (FIG. 13), and interferon-γ (FIG. 14) concentrations in mice of the HSL group were significantly higher than immunoglobulin A, immunoglobulin G, and interferon-γ concentrations in mice of the HFD group, indicating that Staphylococcus lentus could enhance the humoral immunity and cellular immunity in HFD-induced mice, thereby improving the immunity of mice.

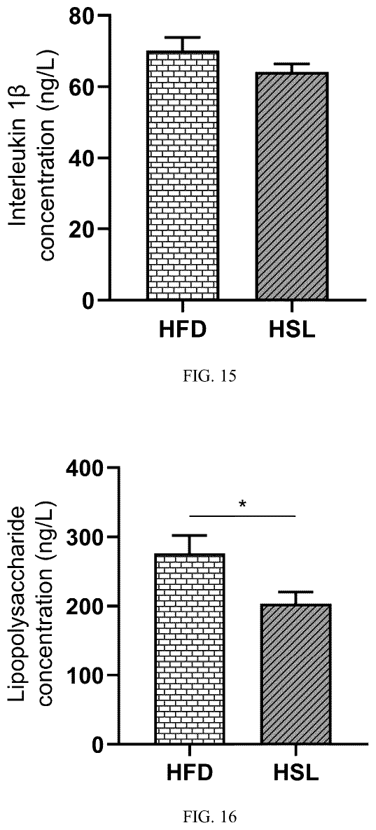

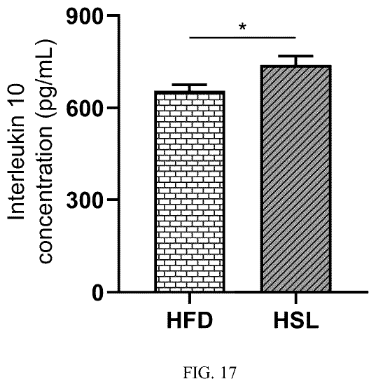

Serum concentrations of pro-inflammatory factors interleukin 1β and lipopolysaccharides and an anti-inflammatory factor interleukin 10 in mice were detected according to steps of ELISA kits to investigate the influence of Staphylococcus lentus on the inflammation in HFD-induced mice. Results were shown in FIG. 15 to FIG. 17. Staphylococcus lentus had no influence on the pro-inflammatory factor interleukin 1β (FIG. 15), but could inhibit the release of the pro-inflammatory factor lipopolysaccharides (P<0.05) (FIG. 16) and could promote the synthesis and secretion of the anti-inflammatory factor interleukin 10 (FIG. 17). In summary, compared with the HFD group, the addition of Staphylococcus lentus resulted in the decrease in a concentration of the pro-inflammatory factor and the increase in a concentration of the anti-inflammatory factor, indicating that Staphylococcus lentus could improve the inflammation induced by HFD.

The above are merely preferred specific embodiments of the present disclosure, but the protection scope of the present disclosure is not limited thereto. Any equivalent replacement or modification made by a person skilled in the art within the technical scope of the present disclosure according to the technical solutions of the present disclosure and inventive concepts thereof shall fall within the protection scope of the present disclosure.

1. A use of Staphylococcus lentus in a preparation of a composition, wherein the composition is a food, a health care product, or a drug, and the composition is used to prevent or treat insulin resistance or type 2 diabetes.

2. The use according to claim 1, wherein the Staphylococcus lentus is a strain deposited in Guangdong Microbial Culture Collection Center (GDMCC), with an accession number of GDMCC 1.247.

3. The use according to claim 1, wherein the Staphylococcus lentus is in a viable cell form.

4. The use according to claim 1, wherein when the composition is the drug, the Staphylococcus lentus serves as a major active ingredient.

5. The use according to claim 1, wherein the type 2 diabetes is caused by the insulin resistance.

6. The use according to claim 1, wherein the insulin resistance is caused by a high-fat diet (HFD).

7. The use according to claim 1, wherein the use further comprises improving one or more of weight gain, impaired glucose tolerance, immune weakness, and inflammation symptoms.

8. The use according to claim 1, wherein when the composition is the drug, a dosage form of the drug is a liquid preparation or a solid preparation.

9. The use according to claim 1, wherein when the composition is the drug, the drug is an oral drug.

10. The use according to claim 1, wherein the drug is administered in a gastrointestinal administration mode.
